(19) United States
(12) Patent Application Publication
(10) Pub. No.: US 2009/0131407 A1
(43) Pub. Date: May 21, 2009

(54) TETRACYCLIC KINASE INHIBITORS

(76) Inventors: Brian Safina, Redwood City, CA (US); Thomas E. Rawson, Mountain View, CA (US); Ignacio Aliagas-Martin, San Francisco, CA (US); Bing-Yan Zhu, Palo Alto, CA (US)

Correspondence Address: GENENTECH, INC., 1 DNA WAY, SOUTH SAN FRANCISCO, CA, 94080, US

(21) Appl. No.: 12/096,586

(22) PCT Filed: Dec. 14, 2006
(86) PCT No.: PCT/US2006/062112

§ 371 Date: Oct. 17, 2008

Related U.S. Application Data
(60) Provisional application No. 60/751,234, filed on Dec. 16, 2005.

(51) Int. Cl. A61K 31/55; C12N 9/99; A61K 31/4188; A61K 31/4196; C07D 487/14; C07D 495/14; A61P 35/00

(52) U.S. Cl. 514/215; 435/184; 514/393; 514/383; 540/578; 548/262.4; 548/302.1

(57) ABSTRACT

The invention provides novel kinase inhibitors that are useful as therapeutic agents for example in the treatment malignancies where the compounds have the general formula (I): I wherein X, Y, Z, R₁, R₂, R₃, Rₐ, R_{b}, and n are as described herein.

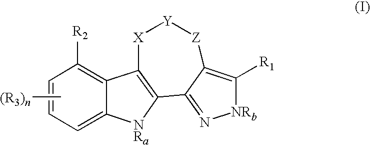

### FIELD OF THE INVENTION

The present invention relates to organic compounds useful for therapy and/or prophylaxis in a mammal, and in particular to inhibitors of kinases useful for treating cancers.

### BACKGROUND OF THE INVENTION

An important class of enzymes that has been the subject of extensive study is protein kinases which are involved in a majority of cellular signaling pathways affecting cell proliferation, migration, differentiation, and metabolism. Kinases function by removing a phosphate group from ATP and phosphorylating hydroxyl groups on serine, threonine and tyrosine amino acid residues of proteins in response to a stimulus such as environmental and chemical stress signals (e.g. osmotic shock, heat shock, ultraviolet radiation, bacterial endotoxin), cytokines (e.g., interleukin-1 and tumor necrosis factor alpha), and growth factors (e.g. granulocyte macrophage-colony-stimulating factor, transforming growth factor, fibroblast growth factor). Many diseases are associated with abnormal cellular responses triggered by protein kinase-mediated events. These diseases include autoimmune diseases, inflammatory diseases, bone diseases, metabolic diseases, neurological and neurodegenerative diseases, cancer, cardiovascular diseases, allergies and asthma, Alzheimer's disease and hormone-related diseases. Accordingly, there has been a substantial effort in medicinal chemistry to find inhibitors of protein kinase that are effective as therapeutic agents.

Aurora kinase is a family of serine/threonine kinases that are essential for cell proliferation. The three known mammalian family members, Aurora-A (also referred to as Aurora-2, Aur-2, STK-15), Aurora-B (also referred to as Aurora-1, Aur-1 and STK-12) and Aurora-C (also referred to as STK-13), are highly homologous proteins responsible for chromosome segregation, mitotic spindle function and cytokinesis. (Bischoff, J. R. & Plowman, G. D., Trends in Cell Biology 9:454, 1999; Giet R. and Prigent, C. Journal of Cell Science 112:3591, 1999; Nigg, E. A., Nat. Rev. Mol. Cell. Biol. 2:21, 2001; Adams, R. R. Carmena, M. and Earnshaw, W. C., Trends in Cell Biology 11:49, 2001). Aurora kinase expression is low or undetectable in resting cells, with expression and activity peaking during the G2 and mitotic phases in cycling cells. In mammalian cells, proposed substrates for Aurora kinases include histone H3, a protein involved in chromosome condensation, centromere protein A (CENP-A), myosin II regulatory light chain, protein phosphatase 1 (PP1), TPX2, all of which are required for cell division. Aurora-A plays a role in the cell cycle by controlling the accurate segregation of chromosomes during mitosis and misregulation thereof can lead to cellular proliferation and other abnormalities.

Since its discovery in 1997 the mammalian Aurora kinase family has been closely linked to tumorigenesis due to its effect on genetic stability. Cells with elevated levels of this kinase contain multiple centrosomes and multipolar spindles, and rapidly become aneuploid. Indeed, a correlation between amplification of the Aurora-A locus and chromosomal instability in mammary and gastric tumors has been observed. (Miyoshi, Y., Iwao, K., Egawa, C., and Noguchi, S. Int. J. Cancer 92:370, 2001; Sakakura, C. et al. British Journal of Cancer 84:824, 2001). Moreover, Aurora-A overexpression has been shown to transforms rodent fibroblasts (Bischoff, J. R., et al. EMBO J. 17:3052, 1998).

The Aurora kinases have been reported to be overexpressed in a wide range of human tumors. Elevated expression of Aurora-A has been detected in over 50% of colorectal, ovarian and gastric cancers, and in 94% of invasive duct adenocarcinomas of the breast. Amplification and/or overexpression of Aurora-A have also been reported in renal, cervical, neuroblastoma, melanoma, lymphoma, bladder, pancreatic and prostate tumors and is associated with aggressive clinical behavior. For example, amplification of the aurora-A locus (20q13) correlates with poor prognosis for patients with node-negative breast cancer (Isola, J. J., et al. American Journal of Pathology 147:905, 1995). Aurora-B is highly expressed in multiple human tumor cell lines, including colon, breast, lung, melanoma, kidney, ovary, pancreas, CNS, gastric tract and leukemias (Tatsuka et al 1998 58, 4811-4816; Katayama et al., Gene 244:1). Also, levels of Aurora-B enzyme have been shown to increase as a function of Duke's stage in primary colorectal cancers (Katayama, H. et al. Journal of the National Cancer Institute 91:1160, 1999). Aurora-C, which is normally only found in testis, is also overexpressed in a high percentage of primary colorectal cancers and in a variety of tumor cell lines including cervical adenocarinoma and breast carcinoma cells (Kimura, M., et al., Journal of Biological Chemistry 274:7334, 1999; Takahashi, T., et al., Jpn. J. Cancer Res. 91:1007-1014, 2000).

Based on the known function of the Aurora kinases, inhibition of their activity will disrupt mitosis leading to cell cycle arrest halting cellular proliferation and therefore will slow tumor growth in a wide range of cancers.

### SUMMARY OF THE INVENTION

In one aspect of the present invention there is provided novel inhibitors of Aurora kinases having the general formula (I)

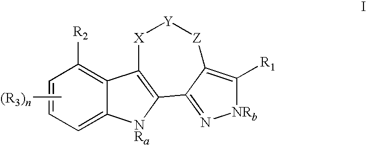

wherein
X, Y and Z are independently absent, CR₄R_{4′}, NR₅, S, SO, SO₂ or O; or X and Y together are CR₄═CR₄; or Y and Z together are CR₄═CR₄; wherein at least one of X, Y and Z is NR₅, S, SO, SO₂ or O;
Rₐ and R_{b} are independently H or a protecting group;
R₁ is H, hydroxyl, halogen, amino, or R₁ is alkyl, acyl, alkoxy or alkylthio optionally substituted with hydroxyl, halogen, oxo, thione, amino, carboxyl and alkoxy;
R₂ is H, halogen, hydroxyl, mercapto, amino, alkyl, a carbocycle or a heterocycle, wherein said alkyl, carbocycle and heterocycle are optionally substituted with halogen, hydroxyl, mercapto, amino, carboxyl, alkyl, a carbocycle or a heterocycle and wherein one or more CH₂ groups of an alkyl group is optionally replaced with —O—, —S—, —S(O)—, S(O)₂, —N(R₅)—, —C(O)—, —C(S)—, —C(O)—NR₅—, —NR₅—C(O)—, —SO₂—NR₅—, —NR₅—SO₂—, —NR₅—C(O)—NR₅—, —C(O)—O— or —O—C(O)—;
R₃ is halogen, hydroxyl, mercapto, amino, alkyl, a carbocycle or a heterocycle, wherein said alkyl, carbocycle and heterocycle are optionally substituted with halogen, hydroxyl, mercapto, amino, carboxyl, alkyl, a carbocycle or a heterocycle and wherein one or more CH₂ groups of an alkyl group is optionally replaced with —O—, —S—, —S(O)—, S(O)₂, —N(R₅)—, —C(O)—, —C(S)—, —C(O)—NR₅—, —NR₅—C(O)—, —SO₂—NR₅—, —NR₅—SO₂—, —NR₅—C(O)—NR₅—, —C(O)—O— or —O—C(O)—;
R₄ and R₄′ are independently H, hydroxyl, halogen, amino, alkyl, a carbocycle or a heterocycle, or R₄ and R_{4′} together form oxo, thione, a carbocycle or heterocycle, wherein said alkyl, carbocycles and heterocycles are optionally substituted with halogen, hydroxyl, carboxyl, amino, alkyl, a carbocycle or a heterocycle and wherein one or more CH₂ groups of an alkyl group is optionally replaced with —O—, —S—, —S(O)—, S(O)₂, —N(R₅)—, —C(O)—, —C(O)—NR₅—, —NR₅—C(O)—, —SO₂—NR₅—, —NR₅—SO₂—, —NR₅—C(O)—NR₅—, —C(O)—O— or —O—C(O)—;
R₅ is H, alkyl, a carbocycle or a heterocycle wherein one or more CH₂ or CH groups of said alkyl is optionally replaced with —O—, —S—, —S(O)—, S(O)₂, —NH—, or —C(O)—; and said alkyl, carbocycle and heterocycle is optionally substituted with hydroxyl, alkoxy, acyl, halogen, mercapto, oxo, carboxyl, acyl, halo-substituted alkyl, amino, cyano nitro, amidino, guanidino an optionally substituted carbocycle or an optionally substituted heterocycle;
n is 0 to 3;
and salts and solvates thereof.

In another aspect of the invention, there are provided pharmaceutical compositions comprising compounds of formula I and a carrier, diluent or excipient.

In another aspect of the invention, there is provided a method for inhibiting the signaling of an Aurora kinase in a cell comprising contacting said Aurora kinase with a compound of formula I.

In another aspect of the invention, there is provided a method for treating a disease or condition in a mammal associated with the signaling of an Aurora kinase, comprising administering to said mammal an effective amount of a compound of formula I.

### DETAILED DESCRIPTION OF THE PREFERRED EMBODIMENTS

“Acyl” means a carbonyl containing substituent represented by the formula —C(O)—R in which R is H, alkyl, a carbocycle, a heterocycle, carbocycle-substituted alkyl or heterocycle-substituted alkyl wherein the alkyl, alkoxy, carbocycle and heterocycle are as defined herein. Acyl groups include alkanoyl (e.g. acetyl), aroyl (e.g. benzoyl), and heteroaroyl.

“Alkyl” means a branched or unbranched, saturated or unsaturated (i.e. alkenyl, alkynyl) aliphatic hydrocarbon group, having up to 12 carbon atoms unless otherwise specified. When used as part of another term, for example “alkylamino”, the alkyl portion may be a saturated hydrocarbon chain, however also includes unsaturated hydrocarbon carbon chains such as “alkenylamino” and “alkynylamino. Examples of particular alkyl groups are methyl, ethyl, n-propyl, isopropyl, n-butyl, iso-butyl, sec-butyl, tert-butyl, n-pentyl, 2-methylbutyl, 2,2-dimethylpropyl, n-hexyl, 2-methylpentyl, 2,2-dimethylbutyl, n-heptyl, 3-heptyl, 2-methylhexyl, and the like. The terms “lower alkyl” “C₁-C₄ alkyl” and “alkyl of 1 to 4 carbon atoms” are synonymous and used interchangeably to mean methyl, ethyl, 1-propyl, isopropyl, cyclopropyl, 1-butyl, sec-butyl or t-butyl. Unless specified, substituted, alkyl groups may contain one, for example two, three or four substituents which may be the same or different. Examples of substituents are, unless otherwise defined, halogen, amino, hydroxyl, protected hydroxyl, mercapto, carboxy, alkoxy, nitro, cyano, amidino, guanidino, urea, sulfonyl, sulfinyl, aminosulfonyl, alkylsulfonylamino, arylsulfonylamino, aminocarbonyl, acylamino, alkoxy, acyl, acyloxy, a carbocycle, a heterocycle. Examples of the above substituted alkyl groups include, but are not limited to; cyanomethyl, nitromethyl, hydroxymethyl, trityloxymethyl, propionyloxymethyl, aminomethyl, carboxymethyl, carboxyethyl, carboxypropyl, alkyloxycarbonylmethyl, allyloxycarbonylaminomethyl, carbamoyloxymethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichloro(n-butyl), 2-amino(iso-propyl), 2-carbamoyloxyethyl and the like. The alkyl group may also be substituted with a carbocycle group. Examples include cyclopropylmethyl, cyclobutylmethyl, cyclopentylmethyl, and cyclohexylmethyl groups, as well as the corresponding -ethyl, -propyl, -butyl, -pentyl, -hexyl groups, etc. Substituted alkyls include substituted methyls e.g. a methyl group substituted by the same substituents as the “substituted Cₙ-Cₘ alkyl” group. Examples of the substituted methyl group include groups such as hydroxymethyl, protected hydroxymethyl (e.g. tetrahydropyranyloxymethyl), acetoxymethyl, carbamoyloxymethyl, trifluoromethyl, chloromethyl, carboxymethyl, bromomethyl and iodomethyl.

“Amidine” means the group —C(NH)—NHR wherein R is H or alkyl or aralkyl. A particular amidine is the group —NH—C(NH)—NH₂.

“Amino” means primary (i.e. —NH₂), secondary (i.e. —NRH) and tertiary (i.e. —NRR) amines wherein R is H, alkyl (e.g. methyl, ethyl, propyl), a carbocycle (e.g. cyclohexyl, phenyl), a heterocycle (e.g. piperidinyl, piperizinyl, pyridinyl) or aralkyl (e.g. benzyl). Particular secondary and tertiary amines are alkylamine, dialkylamine, arylamine, diarylamine, aralkylamine and diaralkylamine wherein the alkyl is as herein defined and optionally substituted. Particular secondary and tertiary amines are methylamine, ethylamine, propylamine, isopropylamine, phenylamine, benzylamine dimethylamine, diethylamine, dipropylamine and diisopropylamine.

“Amino-protecting group” as used herein refers to a derivative of the groups commonly employed to block or protect an amino group while reactions are carried out on other functional groups on the compound. Examples of such protecting groups include carbamates, amides, alkyl and aryl groups, imines, as well as many N-heteroatom derivatives which can be removed to regenerate the desired amine group. Particular amino protecting groups are Boc, Fmoc and Cbz. Further examples of these groups are found in T. W. Greene and P. G. M. Wuts, “Protective Groups in Organic Synthesis”, 2^{nd} ed., John Wiley & Sons, Inc., New York, N.Y., 1991, chapter 7; E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981. The term “protected amino” refers to an amino group substituted with one of the above amino-protecting groups.

“Aryl” when used alone or as part of another term means a carbocyclic aromatic group whether or not fused having the number of carbon atoms designated or if no number is designated, up to 14 carbon atoms. Particular aryl groups are phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). A particular aryl is phenyl. Substituted phenyl or substituted aryl means a phenyl group or aryl group substituted with one, two, three, four or five, for example 1-2, 1-3 or 1-4 substituents chosen, unless otherwise specified, from halogen (F, Cl, Br, I), hydroxy, protected hydroxy, cyano, nitro, alkyl (for example C₁-C₆ alkyl), alkoxy (for example C₁-C₆ alkoxy), benzyloxy, carboxy, protected carboxy, carboxymethyl, protected carboxymethyl, hydroxymethyl, protected hydroxymethyl, aminomethyl, protected aminomethyl, trifluoromethyl, alkylsulfonylamino, alkylsulfonylaminoalkyl, arylsulfonylamino, arylsulonylaminoalkyl, heterocyclylsulfonylamino, heterocyclylsulfonylaminoalkyl, heterocyclyl, aryl, or other groups specified. One or more methyne (CH) and/or methylene (CH₂) groups in these substituents may in turn be substituted with a similar group as those denoted above. Examples of the term “substituted phenyl” includes but is not limited to a mono- or di(halo)phenyl group such as 2-chlorophenyl, 2-bromophenyl, 4-chlorophenyl, 2,6-dichlorophenyl, 2,5-dichlorophenyl, 3,4-dichlorophenyl, 3-chlorophenyl, 3-bromophenyl, 4-bromophenyl, 3,4-dibromophenyl, 3-chloro-4-fluorophenyl, 2-fluorophenyl and the like; a mono- or di(hydroxy)phenyl group such as 4-hydroxyphenyl, 3-hydroxyphenyl, 2,4-dihydroxyphenyl, the protected-hydroxy derivatives thereof and the like; a nitrophenyl group such as 3- or 4-nitrophenyl; a cyanophenyl group, for example, 4-cyanophenyl; a mono- or di(lower alkyl)phenyl group such as 4-methylphenyl, 2,4-dimethylphenyl, 2-methylphenyl, 4-(iso-propyl)phenyl, 4-ethylphenyl, 3-(n-propyl)phenyl and the like; a mono or di(alkoxy)phenyl group, for example, 3,4-dimethoxyphenyl, 3-methoxy-4-benzyloxyphenyl, 3-methoxy-4-(1-chloromethyl)benzyloxy-phenyl, 3-ethoxyphenyl, 4-(isopropoxy)phenyl, 4-(t-butoxy)phenyl, 3-ethoxy-4-methoxyphenyl and the like; 3- or 4-trifluoromethylphenyl; a mono- or dicarboxyphenyl or (protected carboxy)phenyl group such 4-carboxyphenyl; a mono- or di(hydroxymethyl)phenyl or (protected hydroxymethyl)phenyl such as 3-(protected hydroxymethyl)phenyl or 3,4-di(hydroxymethyl)phenyl; a mono- or di(aminomethyl)phenyl or (protected aminomethyl)phenyl such as 2-(aminomethyl)phenyl or 2,4-(protected aminomethyl)phenyl; or a mono- or di(N-(methylsulfonylamino))phenyl such as 3-(N-methylsulfonylamino))phenyl. Also, the term “substituted phenyl” represents disubstituted phenyl groups where the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl, and the like, as well as trisubstituted phenyl groups where the substituents are different, for example 3-methoxy-4-benzyloxy-6-methyl sulfonylamino, 3-methoxy-4-benzyloxy-6-phenyl sulfonylamino, and tetrasubstituted phenyl groups where the substituents are different such as 3-methoxy-4-benzyloxy-5-methyl-6-phenyl sulfonylamino. Particular substituted phenyl groups include the 2-chlorophenyl, 2-aminophenyl, 2-bromophenyl, 3-methoxyphenyl, 3-ethoxy-phenyl, 4-benzyloxyphenyl, 4-methoxyphenyl, 3-ethoxy-4-benzyloxyphenyl, 3,4-diethoxyphenyl, 3-methoxy-4-benzyloxyphenyl, 3-methoxy-4-(1-chloromethyl)benzyloxy-phenyl, 3-methoxy-4-(1-chloromethyl)benzyloxy-6-methyl sulfonyl aminophenyl groups. Fused aryl rings may also be substituted with any, for example 1, 2 or 3, of the substituents specified herein in the same manner as substituted alkyl groups.

“Carbocyclyl”, “carbocyclylic”, “carbocycle” and “carbocyclo” alone and when used as a moiety in a complex group such as a carbocycloalkyl group, refers to a mono-, bi-, or tricyclic aliphatic ring having 3 to 14 carbon atoms, for example 3 to 7 carbon atoms, which may be saturated or unsaturated, aromatic or non-aromatic. Particular saturated carbocyclic groups are cyclopropyl, cyclobutyl, cyclopentyl and cyclohexyl groups. A particular saturated carbocycle is cyclopropyl. Another particular saturated carbocycle is cyclohexyl. Particular unsaturated carbocycles are aromatic e.g. aryl groups as previously defined, for example phenyl. The terms “substituted carbocyclyl”, “carbocycle” and “carbocyclo” mean these groups substituted by the same substituents as the “substituted alkyl” group.

“Carboxamide” means the group —C(O)NH₂. Substituted “carboxamide” means a group —C(O)NHR wherein R is the substituent.

“Carboxy-protecting group” as used herein refers to one of the ester derivatives of the carboxylic acid group commonly employed to block or protect the carboxylic acid group while reactions are carried out on other functional groups on the compound. Examples of such carboxylic acid protecting groups include 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxybenzhydryl, 2,2′,4,4′-tetramethoxybenzhydryl, alkyl such as t-butyl or t-amyl, trityl, 4-methoxytrityl, 4,4′-dimethoxytrityl, 4,4′,4″-trimethoxytrityl, 2-phenylprop-2-yl, trimethylsilyl, t-butyldimethylsilyl, phenacyl, 2,2,2-trichloroethyl, beta-(trimethylsilyl)ethyl, beta-(di(n-butyl)methylsilyl)ethyl, p-toluenesulfonylethyl, 4-nitrobenzylsulfonylethyl, allyl, cinnamyl, 1-(trimethylsilylmethyl)prop-1-en-3-yl, and like moieties. The species of carboxy-protecting group employed is not critical so long as the derivatized carboxylic acid is stable to the condition of subsequent reaction(s) on other positions of the molecule and can be removed at the appropriate point without disrupting the remainder of the molecule. In particular, it is important not to subject a carboxy-protected molecule to strong nucleophilic bases, such as lithium hydroxide or NaOH, or reductive conditions employing highly activated metal hydrides such as LiAlH₄. (Such harsh removal conditions are also to be avoided when removing amino-protecting groups and hydroxy-protecting groups, discussed below.) Particular carboxylic acid protecting groups are the alkyl (e.g. methyl, ethyl, t-butyl), allyl, benzyl and p-nitrobenzyl groups. Similar carboxy-protecting groups used in the cephalosporin, penicillin and peptide arts can also be used to protect a carboxy group substituents. Further examples of these groups are found in T. W. Greene and P. G. M. Wuts, “Protective Groups in Organic Synthesis”, 2^{nd} ed., John Wiley & Sons, Inc., New York, N.Y., 1991, chapter 5; E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981, Chapter 5. The term “protected carboxy” refers to a carboxy group substituted with one of the above carboxy-protecting groups.

“Guanidine” means the group —NH—C(NH)—NHR wherein R is H or alkyl or aralkyl. A particular guanidine is the group —NH—C(NH)—NH₂.

“Hydroxy-protecting group” as used herein refers to a derivative of the hydroxy group commonly employed to block or protect the hydroxy group while reactions are carried out on other functional groups on the compound. Examples of such protecting groups include tetrahydropyranyloxy, benzoyl, acetoxy, carbamoyloxy, benzyl, and silylethers (e.g. TBS, TBDPS) groups. Further examples of these groups are found in T. W. Greene and P. G. M. Wuts, “Protective Groups in Organic Synthesis”, 2^{nd} ed., John Wiley & Sons, Inc., New York, N.Y., 1991, chapters 2-3; E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981. The term “protected hydroxy” refers to a hydroxy group substituted with one of the above hydroxy-protecting groups.

“Heterocyclic group”, “heterocyclic”, “heterocycle”, “heterocyclyl”, or “heterocyclo” alone and when used as a moiety in a complex group such as a heterocycloalkyl group, are used interchangeably and refer to any mono-, bi-, or tricyclic, saturated or unsaturated, aromatic (heteroaryl) or non-aromatic ring having the number of atoms designated, generally from 5 to about 14 ring atoms, where the ring atoms are carbon and at least one heteroatom (nitrogen, sulfur or oxygen), for example 1 to 4 heteroatoms. Typically, a 5-membered ring has 0 to 2 double bonds and 6- or 7-membered ring has 0 to 3 double bonds and the nitrogen or sulfur heteroatoms may optionally be oxidized (e.g. SO, SO₂), and any nitrogen heteroatom may optionally be quaternized. Particular non-aromatic heterocycles are morpholinyl (morpholino), pyrrolidinyl, oxiranyl, oxetanyl, tetrahydrofuranyl, 2,3-dihydrofuranyl, 2H-pyranyl, tetrahydropyranyl, thiiranyl, thietanyl, tetrahydrothietanyl, aziridinyl, azetidinyl, 1-methyl-2-pyrrolyl, piperazinyl and piperidinyl. A “heterocycloalkyl” group is a heterocycle group as defined above covalently bonded to an alkyl group as defined above. Particular 5-membered heterocycles containing a sulfur or oxygen atom and one to three nitrogen atoms are thiazolyl, in particular thiazol-2-yl and thiazol-2-yl N-oxide, thiadiazolyl, in particular 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Particular 5-membered ring heterocycles containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Particular benzo-fused 5-membered heterocycles are benzoxazol-2-yl, benzthiazol-2-yl and benzimidazol-2-yl. Particular 6-membered heterocycles contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are a particular group. Substituents for “optionally substituted heterocycles”, and further examples of the 5- and 6-membered ring systems discussed above can be found in W. Druckheimer et al., U.S. Pat. No. 4,278,793. In a particular embodiment, such optionally substituted heterocycle groups are substituted with hydroxyl, alkyl, alkoxy, acyl, halogen, mercapto, oxo (═O), thione (═S) carboxyl, acyl, halo-substituted alkyl, amino, cyano, nitro, amidino and guanidino. It will be understood that by “optionally substituted” is meant that the heterocycle may be substituted with one or more of the same or different substituents specified. Similarly other groups defined herein that are “optionally substituted” may be substituted with one or more of the specified substituents that may be the same or different.

“Heteroaryl” alone and when used as a moiety in a complex group such as a heteroaralkyl group, refers to any mono-, bi-, or tricyclic aromatic ring system having the number of atoms designated where at least one ring is a 5-, 6- or 7-membered ring containing from one to four heteroatoms selected from the group nitrogen, oxygen, and sulfur, and in a particular embodiment at least one heteroatom is nitrogen (Lang's Handbook of Chemistry, supra). Included in the definition are any bicyclic groups where any of the above heteroaryl rings are fused to a benzene ring. Particular heteroaryls incorporate a nitrogen or oxygen heteroatom. The following ring systems are examples of the heteroaryl (whether substituted or unsubstituted) groups denoted by the term “heteroaryl”: thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, thiazinyl, oxazinyl, triazinyl, thiadiazinyl, oxadiazinyl, dithiazinyl, dioxazinyl, oxathiazinyl, tetrazinyl, thiatriazinyl, oxatriazinyl, dithiadiazinyl, imidazolinyl, dihydropyrimidyl, tetrahydropyrimidyl, tetrazolo[1,5-b]pyridazinyl and purinyl, as well as benzo-fused derivatives, for example benzoxazolyl, benzofuryl, benzothiazolyl, benzothiadiazolyl, benzotriazolyl, benzoimidazolyl and indolyl. A particular “heteroaryl” is: 1,3-thiazol-2-yl, 4-(carboxymethyl)-5-methyl-1,3-thiazol-2-yl, 4-(carboxymethyl)-5-methyl-1,3-thiazol-2-yl sodium salt, 1,2,4-thiadiazol-5-yl, 3-methyl-1,2,4-thiadiazol-5-yl, 1,3,4-triazol-5-yl, 2-methyl-1,3,4-triazol-5-yl, 2-hydroxy-1,3,4-triazol-5-yl, 2-carboxy-4-methyl-1,3,4-triazol-5-yl sodium salt, 2-carboxy-4-methyl-1,3,4-triazol-5-yl, 1,3-oxazol-2-yl, 1,3,4-oxadiazol-5-yl, 2-methyl-1,3,4-oxadiazol-5-yl, 2-(hydroxymethyl)-1,3,4-oxadiazol-5-yl, 1,2,4-oxadiazol-5-yl, 1,3,4-thiadiazol-5-yl, 2-thiol-1,3,4-thiadiazol-5-yl, 2-(methylthio)-1,3,4-thiadiazol-5-yl, 2-amino-1,3,4-thiadiazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-1H-tetrazol-5-yl, 1-(methylsulfonic acid)-1H-tetrazol-5-yl sodium salt, 2-methyl-1H-tetrazol-5-yl, 1,2,3-triazol-5-yl, 1-methyl-1,2,3-triazol-5-yl, 2-methyl-1,2,3-triazol-5-yl, 4-methyl-1,2,3-triazol-5-yl, pyrid-2-yl N-oxide, 6-methoxy-2-(n-oxide)-pyridaz-3-yl, 6-hydroxypyridaz-3-yl, 1-methylpyrid-2-yl, 1-methylpyrid-4-yl, 2-hydroxypyrimid-4-yl, 1,4,5,6-tetrahydro-5,6-dioxo-4-methyl-as-triazin-3-yl, 1,4,5,6-tetrahydro-4-(formylmethyl)-5,6-dioxo-as-triazin-3-yl, 2,5-dihydro-5-oxo-6-hydroxy-astriazin-3-yl, 2,5-dihydro-5-oxo-6-hydroxy-as-triazin-3-yl sodium salt, 2,5-dihydro-5-oxo-6-hydroxy-2-methyl-astriazin-3-yl sodium salt, 2,5-dihydro-5-oxo-6-hydroxy-2-methyl-as-triazin-3-yl, 2,5-dihydro-5-oxo-6-methoxy-2-methyl-as-triazin-3-yl, 2,5-dihydro-5-oxo-as-triazin-3-yl, 2,5-dihydro-5-oxo-2-methyl-as-triazin-3-yl, 2,5-dihydro-5-oxo-2,6-dimethyl-as-triazin-3-yl, tetrazolo[1,5-b]pyridazin-6-yl and 8-aminotetrazolo[1,5-b]-pyridazin-6-yl. An alternative group of “heteroaryl” includes; 4-(carboxymethyl)-5-methyl-1,3-thiazol-2-yl, 4-(carboxymethyl)-5-methyl-1,3-thiazol-2-yl sodium salt, 1,3,4-triazol-5-yl, 2-methyl-1,3,4-triazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-11H-tetrazol-5-yl, 1-(methylsulfonic acid)-1H-tetrazol-5-yl sodium salt, 1,2,3-triazol-5-yl, 1,4,5,6-tetrahydro-5,6-dioxo-4-methyl-as-triazin-3-yl, 1,4,5,6-tetrahydro-4-(2-formylmethyl)-5,6-dioxo-as-triazin-3-yl, 2,5-dihydro-5-oxo-6-hydroxy-2-methyl-as-triazin-3-yl sodium salt, 2,5-dihydro-5-oxo-6-hydroxy-2-methyl-as-triazin-3-yl, tetrazolo[1,5-b]pyridazin-6-yl, and 8-aminotetrazolo[1,5-b]pyridazin-6-yl. Heteroaryl groups are optionally substituted as described for heterocycles.

“Inhibitor” means a compound which reduces or prevents the phosphorylation of Aurora kinases or which reduces or prevents the signaling of Aurora kinase. Alternatively, “inhibitor” means a compound which arrests cells in the G2 phase of the cell cycle.

“Pharmaceutically acceptable salts” include both acid and base addition salts. “Pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, carbonic acid, phosphoric acid and the like, and organic acids may be selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, malonic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicylic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Particularly base addition salts are the ammonium, potassium, sodium, calcium and magnesium salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly organic non-toxic bases are isopropylamine, diethylamine, ethanolamine, trimethamine, dicyclohexylamine, choline, and caffeine.

The present invention provides novel compounds having the general formula I:

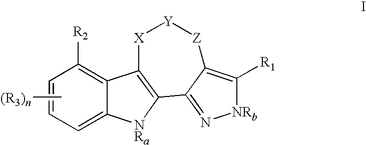

wherein X, Y, Z, R₁, R₂, R₃, Rₐ, R_{b}, and n are as described herein.

X, Y and Z are independently absent, CR₄R_{4′}, NR₅, S, SO, SO₂ or O; or X and Y together are CR₄═CR₄; or Y and Z together are CR₄═CR₄; wherein at least one of X, Y and Z is NR₅, S, SO, SO₂ or O. By absent is meant that X, Y or Z is not present, for example when Y is absent X and Z are bonded and the ring in which they are incorporated has six members. In a particular embodiment X is CR₄R_{4′}, Y is S and Z is CR₄R_{4′}. In a particular embodiment X is CR₄R_{4′}, Y is NR₅, and Z is CR₄R_{4′}. In a particular embodiment X is S, Y is CR₄R_{4′}, and Z is CR₄R_{4′}.

Rₐ and R_{b} are independently H or a protecting group. In a particular embodiment Rₐ and R_{b} are both the same or different acid labile amino protecting group. In a particular embodiment Rₐ and R_{b} are the same or different acyloxy group, for example —OC(O)R_{c}, wherein R_{c} is alkyl, aryl or aralkyl. In a particular embodiment R_{c} is alkyl, for example, methyl, ethyl, propyl, butyl, t-butyl (i.e. forming a t-Boc group). In a particular embodiment Rₐ and R_{b} are both t-Boc. In a particular embodiment Rₐ and R_{b} are both H.

R₁ is H, hydroxyl, halogen, amino, or is alkyl, acyl, alkoxy or alkylthio optionally substituted with hydroxyl, halogen, oxo (═O), thione (═S), amino, carboxyl and alkoxy. In a particular embodiment, R₁ is H. In a particular embodiment R₁ is alkyl. In a particular embodiment R₁ is methyl.

R₂ is H, halogen, hydroxyl, mercapto, amino, alkyl, a carbocycle or a heterocycle, wherein said alkyl, carbocycle and heterocycle are optionally substituted with halogen, hydroxyl, mercapto, amino, carboxyl, alkyl, a carbocycle or a heterocycle and wherein one or more CH₂ groups of an alkyl group is optionally replaced with —O—, —S—, —S(O)—, S(O)₂, —N(R₅)—, —C(O)—, —C(S)—, —C(O)—NR₅—, —NR₅—C(O)—, —SO₂—NR₅—, —NR₅—SO₂—, —NR₅—C(O)—NR₅—, —C(O)—O— or —O—C(O)—. It will be understood that a CH₂ group may be replaced at any position along an alkyl chain including a terminal CH₂ group in which case the replacing group is attached to the preceding carbon atom and a following hydrogen. By way of example, CH₂ groups in a propyl substituent may be replaced with —O— in the following different ways: —O—CH₂—CH₃, —CH₂—O—CH₃ or —CH₂—CH₂—O—H. It is also understood that “an alkyl group” refers to any alkyl portion of a group in the definition of R₂. In a particular embodiment R₂ is H, or an optionally substituted alkyl, carbocycle or heterocycle wherein the substituents are halogen, hydroxyl, amino and mercapto and wherein one or more CH₂ groups of said alkyl group is optionally replaced with —O—, —S—, —S(O)—, S(O)₂, —N(R₅)—, —C(O)—, —C(S)—, —C(O)—NR₅—, —NR₅—C(O)—, —SO₂—NR₅—, —NR₅—SO₂—, —NR₅—C(O)—NR₅—, —C(O)—O— or —O—C(O)—. In a particular embodiment R₂ is an optionally substituted carbocycle or heterocycle. In a particular embodiment R₂ is an optionally substituted aryl or heteroaryl ring. In a particular embodiment R₂ is H or alkyl wherein one more CH₂ groups of said alkyl moiety is optionally replaced with —O—, —S—, —S(O)—, S(O)₂, —N(R₅)—, —C(O)—, —C(S)—, —C(O)—NR₅—, —NR₅—C(O)—, —SO₂—NR₅—, —NR₅—SO₂—, —NR₅—C(O)—NR₅—, —C(O)—O— or —O—C(O)—. In a particular embodiment R₂ is an optionally substituted aryl such as phenyl. In a particular embodiment R₂ is —NH₂, carboxyl, 2-carboxyethenyl, carboxamide, aminocarboxamide, methylsulfonamide, 1H-1,2,4-triazol-1-yl, 5-amino-1H-1,2,4-triazol-3-yl-thio, 3-mercapto-1H-1,2,4-triazol-1-yl, N-benzyloxycarboxamide, 4-nitro-N-benzyloxycarboxamide, N-hydroxycarboxamide, N-ethoxycarboxamide, morpholinomethanone, 4-hydroxypiperidin-1-ylmethanone, piperidin-1-ylmethanone, N-(tetrahydro-2H-pyran-2-yloxy)carboxamide, 3-amino-1H-pyrazol-1-yl or 1H-imidazol-2-ylthio, 4-methylpiperazin-1-yl)methanone. In a particular embodiment R₂ is H.

R₃ is halogen, hydroxyl, mercapto, amino, alkyl, a carbocycle or a heterocycle, wherein said alkyl, carbocycle and heterocycle are optionally substituted with halogen, hydroxyl, mercapto, amino, carboxyl, alkyl, a carbocycle or a heterocycle and wherein one or more CH₂ groups of an alkyl group is optionally replaced with —O—, —S—, —S(O)—, S(O)₂, —N(R₅)—, —C(O)—, —C(S)—, —C(O)—NR₅—, —NR₅—C(O)—, —SO₂—NR₅—, —NR₅—SO₂—, —NR₅—C(O)—NR₅—, —C(O)—O— or —O—C(O)—. It will be understood that a CH₂ group may be replaced at any position along an alkyl chain including a terminal CH₂ group in which case the replacing group is attached to the preceding carbon atom and a following hydrogen. By way of example, CH₂ groups in a propyl substituent may be replaced with —O— in the following different ways: —O—CH₂—CH₃, —CH₂—O—CH₃ or —CH₂—CH₂—O—H. It is also understood that “an alkyl group” refers to any alkyl portion of a group in the definition of R₃. In a particular embodiment R₃ is alkyl, optionally substituted with halogen, hydroxyl, amino, a carbocycle or a heterocycle and wherein one or more CH₂ groups of an alkyl group is optionally replaced with —O—, —S—, —S(O)—, S(O)₂, —N(R₅)—, —C(O)—, —C(O)—NR₅—, —NR₅—C(O)—, —SO₂—NR₅—, —NR₅—SO₂—, —NR₅—C(O)—NR₅—, —C(O)—O— or —O—C(O)—. In a particular embodiment R₃ is alkyl wherein one or more CH₂ groups of an alkyl group is optionally replaced with —O—, —S—, —S(O)—, S(O)₂, —N(R₅)—, —C(O)—, —C(O)—NR₅—, —NR₅—C(O)—, —SO₂—NR₅—, —NR₅—SO₂—, —NR₅—C(O)—NR₅—, —C(O)—O— or —O—C(O)—. In an embodiment, R₃ is alkyl optionally substituted with oxo, thione, amino, hydroxyl, carboxyl or aminocarbonyl. In a particular embodiment R₃ is —NH₂, carboxyl, 2-carboxyethenyl, carboxamide, aminocarboxamide, methylsulfonamide, 1H-1,2,4-triazol-1-yl, 5-amino-1H-1,2,4-triazol-3-yl-thio, 3-mercapto-1H-1,2,4-triazol-1-yl, N-benzyloxycarboxamide, 4-nitro-N-benzyloxycarboxamide, N-hydroxycarboxamide, N-ethoxycarboxamide, morpholino-methanone, 4-hydroxypiperidin-1-ylmethanone, piperidin-1-ylmethanone, N-(tetrahydro-2H-pyran-2-yloxy)carboxamide, 3-amino-1H-pyrazol-1-yl or 1H-imidazol-2-ylthio, 4-methylpiperazin-1-yl)methanone. In another particular embodiment R₃ is methoxy, methylsulfonyl, 1H-imidazol-1-yl, 1H-1,2,4-triazol-3-yl-thio, 1H-1,2,4-triazol-3-yl-amino, 3-amino-1H-1,2,4-triazol-1-yl or 1-hydroxy-1-(5-methylfuran-2-yl)methyl.

R₄ and R₄′ are independently H, hydroxyl, halogen, amino, alkyl, a carbocycle or a heterocycle, or R₄ and R₄ together form oxo, thione, a carbocycle or heterocycle, wherein said alkyl, carbocycles and heterocycles are optionally substituted with halogen, hydroxyl, carboxyl, amino, alkyl, a carbocycle or a heterocycle and wherein one or more CH₂ groups of an alkyl group is optionally replaced with —O—, —S—, —S(O)—, S(O)₂, —N(R₅)—, —C(O)—, —C(O)—NR₅—, —NR₅—C(O)—, —SO₂—NR₅—, —NR₅—SO₂—, —NR₅—C(O)—NR₅—, —C(O)—O— or —O—C(O)—. It will be understood that a CH₂ group may be replaced at any position along an alkyl chain including a terminal CH₂ group in which case the replacing group is attached to the preceding carbon atom and a following hydrogen. By way of example, CH₂ groups in a propyl substituent may be replaced with —O— in the following different ways: —O—CH₂—CH₃, —CH₂—O—CH₃ or CH₂—CH₂—O—H. It is also understood that “an alkyl group” refers to any alkyl portion of a group in the definition of R₄. In a particular embodiment R₄ and R₄ are independently H, or an optionally substituted alkyl, carbocycle or heterocycle wherein the substituents are halogen, hydroxyl, amino and mercapto and wherein one or more CH₂ groups of said alkyl group is optionally replaced with —O—, —S—, —S(O)—, S(O)₂, —N(R₅)—, —C(O)—, —C(S)—, —C(O)—NR₅—, —NR₅—C(O)—, —SO₂—NR₅—, —NR₅—SO₂—, —NR₅—C(O)—NR₅—, —C(O)—O— or —O—C(O)—. In a particular embodiment R₄ and R₄ are independently an optionally substituted carbocycle or heterocycle. In a particular embodiment R₄ and R_{4′} are independently an optionally substituted aryl or heteroaryl ring. In a particular embodiment R₄ and R_{4′} are independently H or alkyl wherein one more CH₂ groups of said alkyl moiety is optionally replaced with —O—, —S—, —S(O)—, S(O)₂, —N(R₅)—, —C(O)—, —C(S)—, —C(O)—NR₅—, —NR₅—C(O)—, —SO₂—NR₅—, —NR₅—SO₂—, —NR₅—C(O)—NR₅—, —C(O)—O— or —O—C(O)—. In a particular embodiment R₄ is H while R₄ is a group as previously defined other than H. In a particular embodiment R₄ and R_{4′} are independently alkyl such as methyl. In a particular embodiment R₄ and R_{4′} are both H. R₅ is H, alkyl, a carbocycle or a heterocycle wherein one or more CH₂ or CH groups of said alkyl is optionally replaced with —O—, —S—, —S(O)—, S(O)₂, —NH—, or —C(O)—; and said alkyl, carbocycle and heterocycle is optionally substituted with hydroxyl, alkoxy, acyl, halogen, mercapto, oxo, carboxyl, acyl, halo-substituted alkyl, amino, cyano nitro, amidino, guanidino an optionally substituted carbocycle or an optionally substituted heterocycle. In a particular embodiment R₅ is H or alkyl. In a particular embodiment R₅ is H. In a particular embodiment R₅ is alkyl, for example methyl, ethyl or propyl. In an embodiment R₅ is alkoxycarbonyl. In a particular embodiment R₅ is ethyloxycarbonyl.

n is 0 to 3. In an embodiment n is 0 to 2. In an embodiment n is 2. In an embodiment n is 0 to 1. In an embodiment n is 1. In an embodiment n is 0.

Compounds of the invention may contain one or more asymmetric carbon atoms. Accordingly, the compounds may exist as diastereomers, enantiomers or mixtures thereof. The syntheses of the compounds may employ racemates, diastereomers or enantiomers as starting materials or as intermediates. Diastereomeric compounds may be separated by chromatographic or crystallization methods. Similarly, enantiomeric mixtures may be separated using the same techniques or others known in the art. Each of the asymmetric carbon atoms may be in the R or S configuration and both of these configurations are within the scope of the invention.

The invention also encompasses prodrugs of the compounds described above. Suitable prodrugs where applicable include known amino-protecting and carboxy-protecting groups which are released, for example hydrolyzed, to yield the parent compound under physiologic conditions. A particular class of prodrugs are compounds in which a nitrogen atom in an amino, amidino, aminoalkyleneamino, iminoalkyleneamino or guanidino group is substituted with a hydroxy (OH) group, an alkylcarbonyl (—CO—R) group, an alkoxycarbonyl (—CO—OR), an acyloxyalkyl-alkoxycarbonyl (—CO—O—R—O—CO—R) group where R is a monovalent or divalent group and as defined above or a group having the formula —C(O)—O—CP1P2-haloalkyl, where P1 and P2 are the same or different and are H, lower alkyl, lower alkoxy, cyano, halo lower alkyl or aryl. In a particular embodiment, the nitrogen atom is one of the nitrogen atoms of the amidino group of the compounds of the invention. These prodrug compounds are prepared reacting the compounds of the invention described above with an activated acyl compound to bond a nitrogen atom in the compound of the invention to the carbonyl of the activated acyl compound. Suitable activated carbonyl compounds contain a good leaving group bonded to the carbonyl carbon and include acyl halides, acyl amines, acyl pyridinium salts, acyl alkoxides, in particular acyl phenoxides such as p-nitrophenoxy acyl, dinitrophenoxy acyl, fluorophenoxy acyl, and difluorophenoxy acyl. The reactions are generally exothermic and are carried out in inert solvents at reduced temperatures such as −78 to about 50 C. The reactions are usually also carried out in the presence of an inorganic base such as potassium carbonate or sodium bicarbonate, or an organic base such as an amine, including pyridine, triethylamine, etc. One manner of preparing prodrugs is described in U.S. Ser. No. 08/843,369 filed Apr. 15, 1997 (corresponding to PCT publication WO9846576) the contents of which are incorporated herein by reference in their entirety.

Particular compounds of formula I include the following:

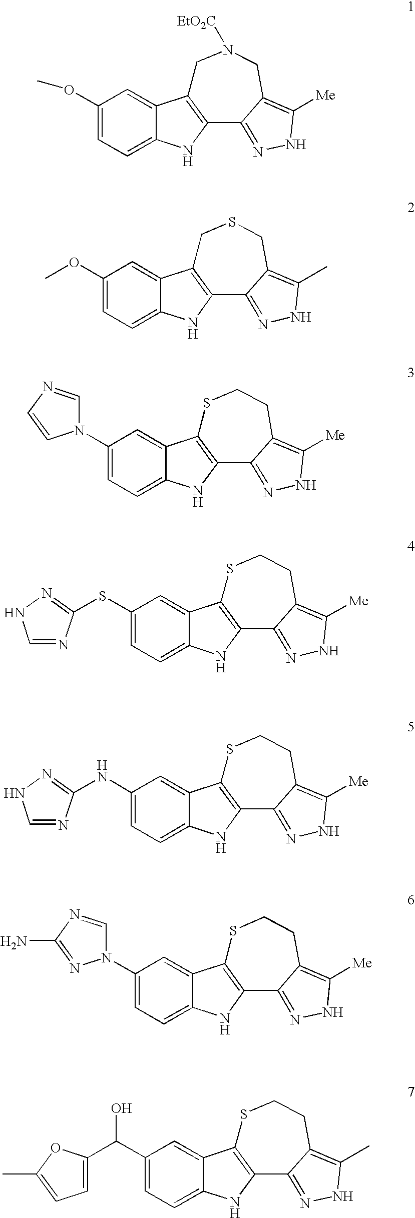

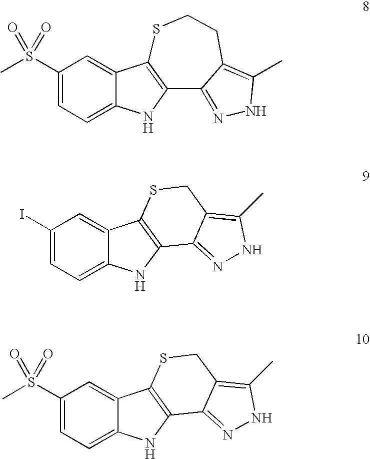

### Synthesis

Compounds of the invention are prepared using standard organic synthetic techniques from commercially available starting materials and reagents. It will be appreciated that synthetic procedures employed in the preparation of compounds of the invention will depend on the particular substituents present in a compound and that various protection and deprotection steps that are standard in organic synthesis may be required but may not be illustrated in the following general schemes. In a particular embodiment compounds of the invention are prepared according to the general synthetic scheme 1.

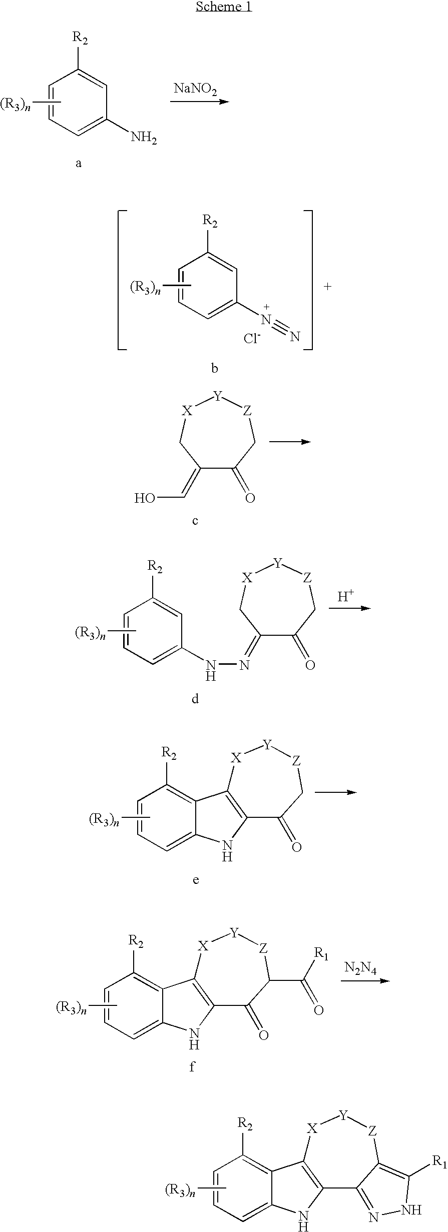

In scheme 1, diazonium salt b is formed by reacting aromatic amine a with sodium nitrite under acidic conditions. The diazonium salt is then coupled to enol c via a Japp-Klingemann reaction to give hydrazone d which undergoes Fischer indole cyclization under acidic conditions to form compound e. Compound e is subsequently reacted with base and the desired R₁-containing electrophile to form beta-ketone compound f which is reacted with hydrazine to form the pyrazole-containing final compound. Suitable R₁-containing electrophiles are anhydrides (R₁, —CO)₂O), nitrites (R₁—CO—CN) and acid halides (R₁, —CO—X).

Compounds of formula I in which X is S and Y and Z are independently CR₄R_{4′}, a bond or together form CR₄═CR₄, may be prepared according to the general scheme 2.

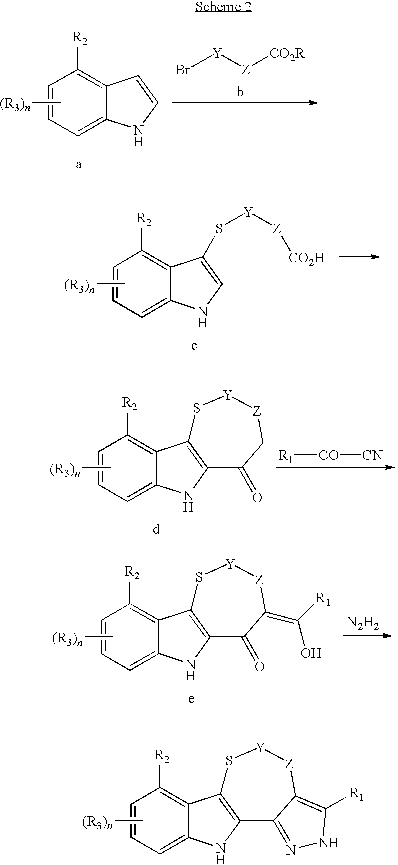

Starting compound a is reacted with thiourea and potassium triiodide followed by the addition of the desired bromo compound b to give intermediate c after a basic workup. Intermediate c is then reacted with polyphosphate ester to give indole-ketone d which is reacted with the desired R₁-containing electrophile R₁—CO—CN to give the enol e. Final pyrazole formation is achieved by reacting enol e with hydrazine.

### Utility

The compounds of the invention inhibit Aurora kinase signaling, in particular the phosphorylation of Aurora kinases. Accordingly, the compounds of the invention are useful for inhibiting all diseases associated with the aberrant signaling, overexpression and/or amplification of Aurora kinases. Alternatively, compounds of the invention are useful for arresting cells in the G2 phase of the cell cycle. More specifically, the compounds can be used for the treatment of cancers associated with aberrant signaling, amplification and/or overexpression of Aurora kinases. Examples of such cancer types include neuroblastoma, intestine carcinoma such as rectum carcinoma, colon carcinoma, familiarly adenomatous polyposis carcinoma and hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, craniopharyngeoma, osteosarcoma, chondrosarcoma, myosarcoma, liposarcoma, fibrosarcoma, Ewing sarcoma and plasmocytoma. In particular, compounds of the invention are useful for treating colorectal, ovarian, gastric, breast (such as invasive duct adenocarcinomas thereof), renal, cervical, melanoma, lymphoma, bladder, pancreatic, prostate, lung, CNS (such as neuroblastoma), cervical and leukemic cancers.

Compounds of the invention may be administered prior to, concomitantly with, or following administration of radiation therapy or cytostatic or antineoplastic chemotherapy. Suitable cytostatic chemotherapy compounds include, but are not limited to (i) antimetabolites, such as cytarabine, fludarabine, 5-fluoro-2′-deoxyuiridine, gemcitabine, hydroxyurea or methotrexate; (ii) DNA-fragmenting agents, such as bleomycin, (iii) DNA-crosslinking agents, such as chlorambucil, cisplatin, cyclophosphamide or nitrogen mustard; (iv) intercalating agents such as adriamycin (doxorubicin) or mitoxantrone; (v) protein synthesis inhibitors, such as L-asparaginase, cycloheximide, puromycin or diphtheria toxin; (Vi) topoisomerase I poisons, such as camptothecin or topotecan; (vii) topoisomerase II poisons, such as etoposide (VP-16) or teniposide; (viii) microtubule-directed agents, such as colcemid, colchicine, paclitaxel, vinblastine or vincristine; (ix) kinase inhibitors such as flavopiridol, staurosporin, STI571 (CPG 57148B) or UCN-01 (7-hydroxystaurosporine); (x) miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; (xi) hormones such as glucocorticoids or fenretinide; (xii) hormone antagonists, such as tamoxifen, finasteride or LHRH antagonists. In a particular embodiment, compounds of the present invention are coadministered with a cytostatic compound selected from the group consisting of cisplatin, doxorubicin, taxol, taxotere and mitomycin C. In a particular embodiment, the cytostatic compound is doxorubicin.

Another class of active compounds which can be used in the present invention are those which are able to sensitize for or induce apoptosis by binding to death receptors (“death receptor agonists”). Such agonists of death receptors include death receptor ligands such as tumor necrosis factor a (TNF-α), tumor necrosis factor β (TNF-β, lymphotoxin-α), LT-β (lymphotoxin-β), TRAIL (Apo2L, DR4 ligand), CD95 (Fas, APO-1) ligand, TRAMP (DR3, Apo-3) ligand, DR6 ligand as well as fragments and derivatives of any of said ligands. In an embodiment, the death receptor ligand is TNF-α. In a particular embodiment, the death receptor ligand is Apo2L/TRAIL. Furthermore, death receptors agonists comprise agonistic antibodies to death receptors such as anti-CD95 antibody, anti-TRAIL-R1 (DR4) antibody, anti-TRAIL-R2 (DR5) antibody, anti-TRAIL-R3 antibody, anti-TRAIL-R4 antibody, anti-DR6 antibody, anti-TNF-R1 antibody and anti-TRAMP (DR3) antibody as well as fragments and derivatives of any of said antibodies.

Compounds of the present invention can be also used in combination with radiation therapy. The phrase “radiation therapy” refers to the use of electromagnetic or particulate radiation in the treatment of neoplasia. Radiation therapy is based on the principle that high-dose radiation delivered to a target area will result in the death of reproducing cells in both tumor and normal tissues. The radiation dosage regimen is generally defined in terms of radiation absorbed dose (rad), time and fractionation, and must be carefully defined by the oncologist. The amount of radiation a patient receives will depend on various consideration but the two most important considerations are the location of the tumor in relation to other critical structures or organs of the body, and the extent to which the tumor has spread. Examples of radiotherapeutic agents are provided in, but not limited to, radiation therapy and is known in the art (Hellman, Principles of Radiation Therapy, Cancer, in Principles I and Practice of Oncology, 24875 (Devita et al., 4th ed., vol 1, 1993). Recent advances in radiation therapy include three-dimensional conformal external beam radiation, intensity modulated radiation therapy (IMRT), stereotactic radiosurgery and brachytherapy (interstitial radiation therapy), the latter placing the source of radiation directly into the tumor as implanted “seeds”. These newer treatment modalities deliver greater doses of radiation to the tumor, which accounts for their increased effectiveness when compared to standard external beam radiation therapy.

Ionizing radiation with beta-emitting radionuclides is considered the most useful for radiotherapeutic applications because of the moderate linear energy transfer (LET) of the ionizing particle (electron) and its intermediate range (typically several millimeters in tissue). Gamma rays deliver dosage at lower levels over much greater distances. Alpha particles represent the other extreme, they deliver very high LET dosage, but have an extremely limited range and must, therefore, be in intimate contact with the cells of the tissue to be treated. In addition, alpha emitters are generally heavy metals, which limits the possible chemistry and presents undue hazards from leakage of radionuclide from the area to be treated. Depending on the tumor to be treated all kinds of emitters are conceivable within the scope of the present invention.

Furthermore, the present invention encompasses types of non-ionizing radiation like e.g. ultraviolet (UV) radiation, high energy visible light, microwave radiation (hyperthermia therapy), infrared (IR) radiation and lasers. In a particular embodiment of the present invention UV radiation is applied.

The invention also includes pharmaceutical compositions or medicaments containing the compounds of the invention and a therapeutically inert carrier, diluent or excipient, as well as methods of using the compounds of the invention to prepare such compositions and medicaments. Typically, the compounds of formula I used in the methods of the invention are formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but may range anywhere from about 3 to about 8. Formulation in an acetate buffer at pH 5 is a suitable embodiment. In an embodiment, the inhibitory compound for use herein is sterile. The compound ordinarily will be stored as a solid composition, although lyophilized formulations or aqueous solutions are acceptable.

The composition of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit Aurora kinase signaling. Such amount may be below the amount that is toxic to normal cells, or the mammal as a whole. Alternatively, “effective amount” of a compound of the invention may be the amount necessary to inhibit the proliferation of cancer cells or the amount required to inhibit the growth of tumors. Generally, the initial pharmaceutically effective amount of the compound of the invention administered parenterally per dose will be in the range of about 0.01-100 mg/kg, for example about 0.1 to 20 mg/kg of patient body weight per day, with the typical initial range of compound used being 0.3 to 15 mg/kg/day. Oral unit dosage forms, such as tablets and capsules, may contain from about 25 to about 1000 mg of the compound of the invention.

The compound of the invention may be administered by any suitable means, including oral, topical, transdermal, parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. An example of a suitable oral dosage form is a tablet containing about 25 mg, 50 mg, 100 mg, 250 mg, or 500 mg of the compound of the invention compounded with about 90-30 mg anhydrous lactose, about 5-40 mg sodium croscarmellose, about 5-30 mg polyvinylpyrrolidone (PVP) K30, and about 1-10 mg magnesium stearate. The powdered ingredients are first mixed together and then mixed with a solution of the PVP. The resulting composition can be dried, granulated, mixed with the magnesium stearate and compressed to tablet form using conventional equipment. An aerosol formulation can be prepared by dissolving the compound, for example 5-400 mg, of the invention in a suitable buffer solution, e.g. a phosphate buffer, adding a tonicifier, e.g. a salt such sodium chloride, if desired. The solution is typically filtered, e.g. using a 0.2 micron filter, to remove impurities and contaminants.

### EXAMPLES

The invention will be more fully understood by reference to the following examples. They should not, however, be construed as limiting the scope of the invention. Reagents and solvents were obtained from commercial sources and used as received.

Abbreviations used herein are as follows:

ACN: acetonitrile;

Chg: cyclohexylglycine;

DCM: dichloromethane

DIPEA: diisopropylethylamine;

DMAP: 4-dimethylaminopyridine;

DME: 1,2-dimethoxyethane;

DMF: dimethylformamide;

DMSO: dimethylsulfoxide

EDC: 1-ethyl-3-(3-dimethylaminopropyl)carbodiimide;

EEDQ: 2-ethoxy-1-ethoxycarbonyl-1,2-dihydroquinoline

LCMS: liquid chromatography mass spectrometry;.

HATU: O-(7-Azobenzotriazol-1-yl)-1,1,3,3-tetramethyluronium hexafluorophosphate;

HOBT: N-Hydroxybenzotriazole

HBTU: 2-(1H-Benzotriazol-1-yl)-1,1,3,3-Tetramethyl-uronium Hexafluorophosphate

HPLC: high performance liquid chromatography;

NBS: N-bromosuccinamide;

TASF: tris(dimethylamino)sulfonium difluorotrimethylsilicate;

TEA: triethylamine;

TFA: trifluoroacetate;

THF: tetrahydrofuran;

### EXAMPLE 1

### synthesis of compound 1

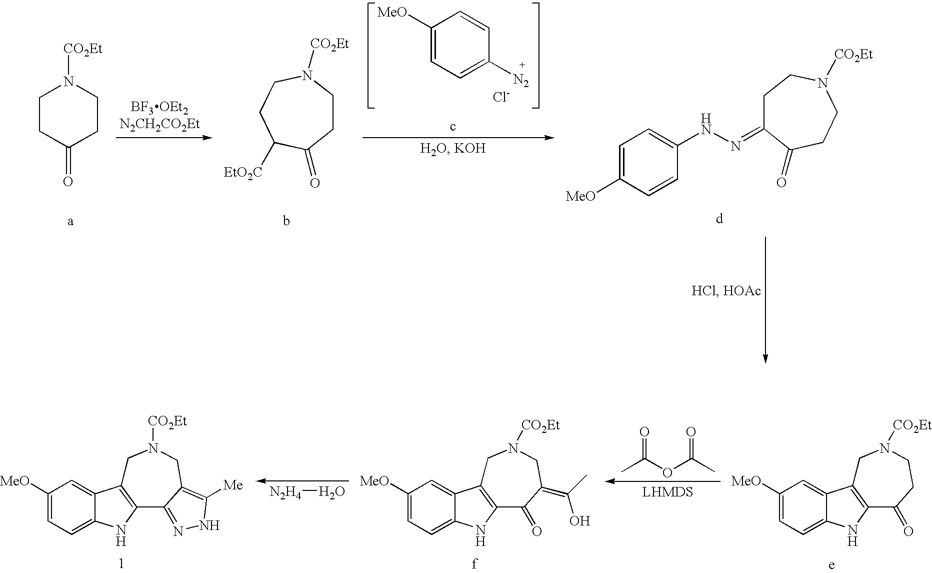

1,5-bis(carbethoxy)perhydroazepine-4-one (b): To a cold solution (−30° C.) of 1-carbethoxypiperidin-4-one (3.0 ml, 26.5 mmol) in anhydrous ether, were simultaneously added a solution of boron trifluoride etherate (3.4 ml, 26.5 mmol) in diethyl ether (2.8 ml) and a solution of ethyl diazodicarboxylate (3.6 ml, 34.5 mmol) in diethyl ether (2.8 ml) over 1.5 hours via a syringe pump. Upon completion of additions, the reaction mixture was allowed to stir for an additional hour at −30° C. and then warmed to room temperature. The reaction mixture was washed with 30% potassium carbonate and the organic phase was dried over Na₂SO₄ and concentrated in vacuo to give b as a crude yellow oil, used directly in the following reaction.

d: To a cold (0° C.) solution of 4-methoxy aniline (1.69 g, 13.8 mmol) and hydrochloric acid (1.72 ml) in H₂O (69 ml) was added an aqueous solution of sodium nitrite (0.95 g, 13.8 mmol, 0.6 M), dropwise over 5 minutes. The resulting solution of the diazonium salt (c), was slowly added to a separate reaction flask containing 1,5-bis(Carbethoxy)perhydroazepine-4-one (b, 13.8 mmol) in H₂O (35 ml), basified with KOH (0.85 g, 15.1 mmol) at 0° C. After stirring for 2-3 hours, the reaction mixture was diluted with 0.1 M HCl (50 ml) and extracted with EtOAc (3×50 ml) and dried over Na₂SO₄. The solvent was removed in vacuo and the residue was subjected to flash chromatography (silica gel, 0→40% EtOAc in hexanes, gradient elution) to afford hydrazone d (1.1 g, 25%).

e: A solution of hydrazone d (1.1 g, 3.3 mmol) in acetic acid (6.6 ml) and hydrochloric acid (0.3 ml) was heated to 80° C. for 1.5 hours. Upon cooling to room temperature, the reaction mixture was diluted with H₂O (10 ml) and extracted with EtOAc (3×20 ml). The organic layer was washed with brine (20 ml) and dried over Na₂SO₄. The solvent was removed in vacuo and the residue was subjected to flash chromatography (silica gel, 0→50% EtOAc in hexanes, gradient elution) to afford indole-ketone (e, 200 mg, 20%).

1: To a cold (0° C.) solution of indole-ketone (e, 200 mg, 0.66 mmol) in THF (4.0 ml) was added LHMDS (2.0 ml, 1.0 M in THF) dropwise. After stirring for 1 hour at 0° C., acetic anhydride was added dropwise and the reaction mixture was allowed to stir for 3 hours while warming to room temperature. The reaction mixture was diluted with H₂O (10 ml) and extracted with EtOAc (3×20 ml). The organic layer was washed with brine (20 ml) and dried over Na₂SO₄. The solvent was removed in vacuo and the crude residue containing f was dissolved in EtOH (3 ml) and hydrazine (1.5 ml) and allowed to stir for 12 hours at room temperature. The reaction mixture was diluted with H₂O (10 ml) and extracted with EtOAc (3×20 ml). The organic layer was washed with brine (20 ml) and dried over Na₂SO₄. The solvent was removed in vacuo and the residue subjected to purification by HPLC, to afford the final compound 1 (47 mg, 21%).

### EXAMPLE 2

### synthesis of compound 2

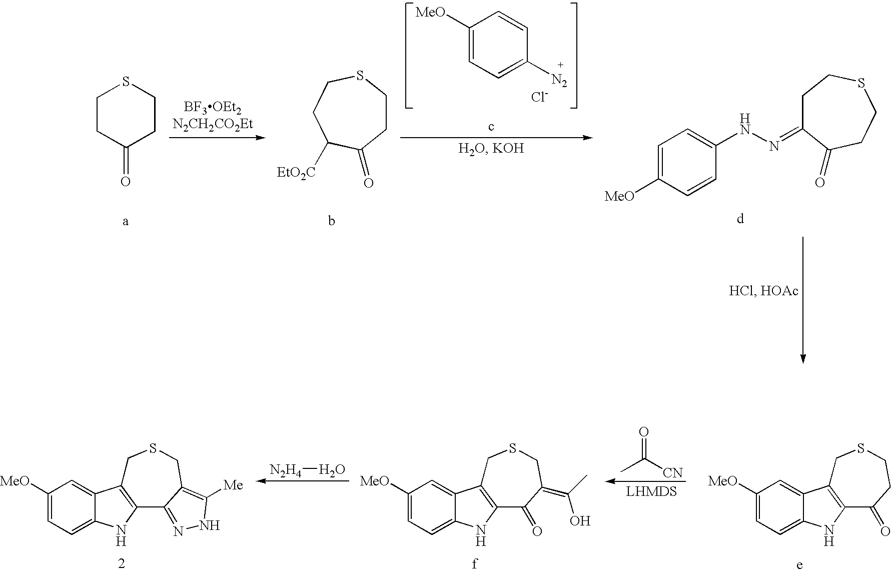

b: To a cold solution (−30° C.) of tetrahydrothiopyran-4-one a (5.0 g, 43.0 mmol) in anhydrous ether, were simultaneously added a solution of boron trifluoride etherate (5.4 ml, 43.0 mmol) in diethyl ether (4.6 ml) and a solution of ethyl diazodicarboxylate (5.8 ml, 55.9 mmol) in diethyl ether (4.6 ml) over 1.5 hours via a syringe pump. Upon completion of additions, the reaction mixture was allowed to stir for an additional hour at −30° C. and then warmed to room temperature. The reaction mixture was washed with 30% potassium carbonate and the organic phase was dried over Na₂SO₄ and concentrated in vacuo to give b as a white solid, used directly in the following reaction.

d: To a cold (0° C.) solution of 4-methoxy aniline c (5.3 g, 43.0 mmol) and hydrochloric acid (5.4 ml) in H₂O (215 ml) was added an aqueous solution of sodium nitrite (2.97 g, 43 mmol, 0.6 M), dropwise over 5 minutes. The resulting solution of the diazonium salt, was slowly added to a separate reaction flask containing b (43 mmol) in H₂O (108 ml), basified with KOH (2.65 g, 47.3 mmol) at 0° C. After stirring for 2-3 hours, the reaction mixture was diluted with 0.1 M HCl (100 ml) and extracted with EtOAc (3×100 ml) and dried over Na₂SO₄. The solvent was removed in vacuo and the residue was subjected to flash chromatography (silica gel, 0→10% EtOAc in hexanes, gradient elution) to afford hydrazone d (2.0 g, 18%).

e: A solution of hydrazone d (2.0 g, 7.6 mmol) in acetic acid (15.2 ml) and hydrochloric acid (0.7 ml) was heated to 80° C. for 1.5 hours. Upon cooling to room temperature, the reaction mixture was diluted with H₂O (20 ml) and extracted with EtOAc (3×40 ml). The organic layer was washed with brine (40 ml) and dried over Na₂SO₄. The solvent was removed in vacuo and the residue was subjected to flash chromatography (silica gel, 0→20% EtOAc in hexanes, gradient elution) to afford indole-ketone e (0.588 g, 31%).

2: To a cold (−78° C.) solution of indole-ketone e (85 mg, 0.34 mmol) in THF (1.7 ml) was added LHMDS (1.0 ml, 1.0 M in THF) dropwise. After stirring for 1 hour at 0° C., pyruvonitrile was added dropwise and the reaction mixture was allowed to stir for 30 minutes at −78° C. The reaction mixture was diluted with H₂O (4 ml) and extracted with EtOAc (3×5 ml). The organic layer was washed with brine (5 ml) and dried over Na₂SO₄. The solvent was removed in vacuo and the crude residue containing f was dissolved in EtOH (1.7 ml) and hydrazine (1.7 ml) and allowed to stir for 12 hours at room temperature. The reaction mixture was diluted with H₂O (5 ml) and extracted with EtOAc (3×10 ml). The organic layer was washed with brine (10 ml) and dried over Na₂SO₄. The solvent was removed in vacuo and the residue subjected to purification by HPLC, to afford final product 2 (34 mg, 35%).

### EXAMPLE 3

### synthesis of compund 8

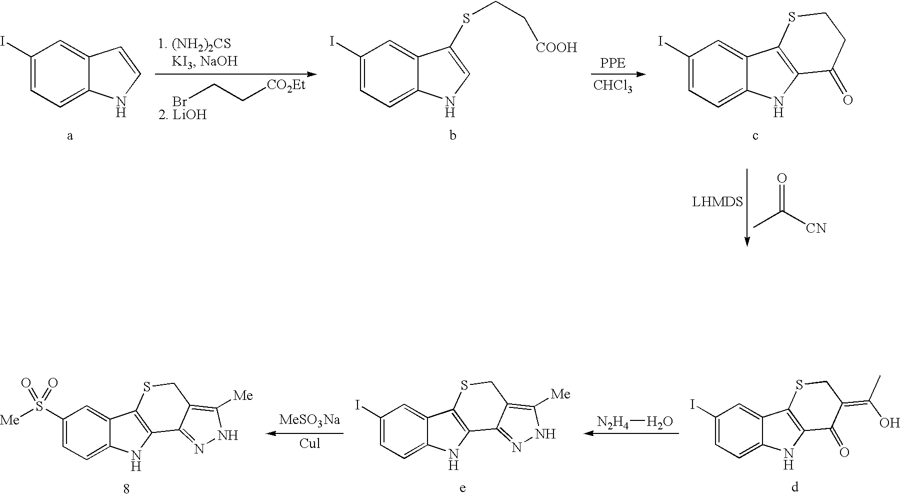

b: A 0.2 M solution of potassium triiodide was prepared by stirring KI (4.9 g, 29.5 mmol) and 12 (5.0 g, 19.7 mmol) in H₂O (100 ml) for 24 hours. This freshly prepared mixture of potassium triiodide was then added dropwise to a solution of 5-iodoindole a (4.0 g, 16.5 mmol) and thiourea (1.5 g, 19.7 mmol) in MeOH (50 ml) at room temperature and stirred for 30 minutes. After filtration, the solution is concentrated under reduced pressure at 45° C., to half of its volume. To this mixture was added an aqueous solution of NaOH (6.6 ml, 10 N) which was heated to 95° C. for 30 minutes. Upon cooling to room temperature, a solution of ethyl bromopropionate (2.1 ml, 16.5 mmol) in diethyl ether (50 ml) was added to the reaction mixture at room temperature and stirred vigorously for 45 minutes. After separation of the organic phase, the aqueous reaction mixture was additionally extracted with diethyl ether (25 ml) and then combined for additional washes with brine (25 ml) and dried over Na₂SO₄. The solvent was removed in vacuo to give a yellow oil, used directly in the next reaction. Lithium hydroxide (0.595 g, 14.2 mmol) was added to the crude ester (2.66 g, 7.09 mmol) dissolved in a 2:2:1 mixture of THF, EtOH and H₂O (14 ml) and heated to 50° C. for 2 hours. After cooling to room temperature, the reaction mixture was quenched with aqueous hydrochloric acid (12 ml, 1 M HCl) and extracted with diethyl ether (25 ml), washed with H₂O (20 ml), brine (20 ml) and dried over Na₂SO₄. The solvent was removed in vacuo, to afford acid b (2.33 g, 95%) as a clear oil.

c: To a solution of acid b (1.62 g, 4.67 mmol) in CHCl₃ (20 ml) was added polyphosphate ester (PPE) (20 ml) and allowed to stir for 1.5 hours. The solution was then poured into ice water and extracted with EtOAc (3×20 ml). The organic layer was further washed with H₂O (20 ml), brine (20 ml) and dried over Na₂SO₄. The solvent was removed in vacuo, and the residue was subjected to flash chromatography (silica gel, 0→50% EtOAc in hexanes, gradient elution) to afford indole-ketone c (0.460 g, 30%) as a yellow oil.

d: To a cold (−78° C.) solution of indole-ketone c (1.0 g, 4.04 mmol) in THF (10 ml) was added LHMDS (12.1 ml, 1.0 M in THF) dropwise. After stirring for 1 hour at 0° C., pyruvonitrile (1.44 ml, 20.2 mmol) was added dropwise and the reaction mixture was allowed to stir for 30 minutes at −78° C. The reaction mixture was diluted with H₂O (10 ml) and extracted with EtOAc (3×20 ml). The organic layer was washed with brine (10 ml) and dried over Na₂SO₄. The solvent was removed in vacuo and the crude residue containing thiopyranoindole d was dissolved in EtOH (20 ml) and hydrazine (5 ml) and allowed to stir for 3 hours at room temperature. The reaction mixture was diluted with 0.1 M HCl (20 ml) and extracted with EtOAc (3×20 ml). The organic layer was washed with brine (20 ml) and dried over Na₂SO₄. The solvent was removed in vacuo and the residue subjected to flash chromatography (silica gel, 0→85% EtOAc in hexanes, gradient elution) to afford pyrazole e (0.460 g, 30%) as a yellow oil.

8: A mixture of pyrazole e (44 mg, 0.12 mmol), CuI (1 mg, 0.006 mmol), Sodium methansulfinate (18 mg, 0.18 mmol), N,N-dimethylethlenediamine (1.27 μl, 0.012 mmol) was dissolved in DMSO (1 ml) and heated to 180° C. over 15 min by microwave. The reaction mixture was diluted with H₂O (1 ml) and extracted with EtOAc (3×2 ml). The organic layer was washed with brine (1 ml) and dried over Na₂SO₄. The solvent was removed in vacuo and the residue subjected to purification by HPLC, to afford final product 8 (6.1 mg, 16%).

### EXAMPLE 4

### synthesis of compund 9

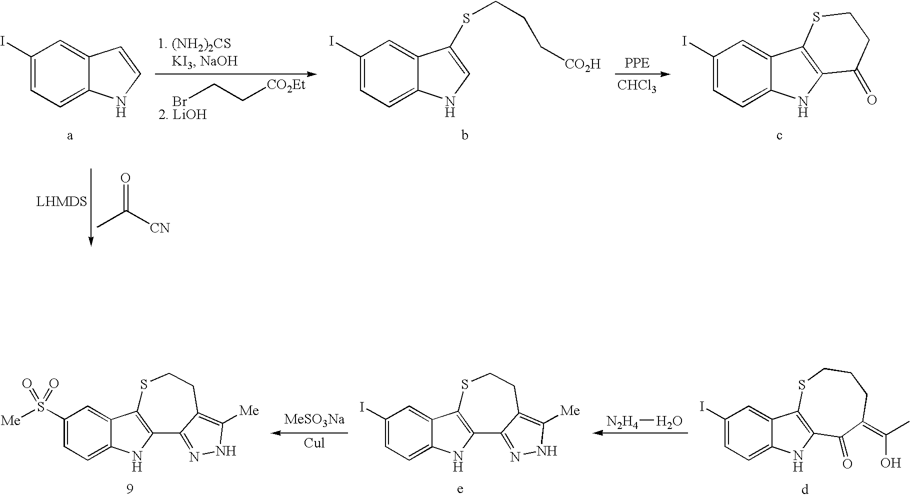

b: A 0.2 M solution of potassium triiodide was prepared by stirring KI (7.25 g, 43.7 mmol) and 12 (7.4 g, 29.1 mmol) in H₂O (146 ml) for 24 hours. This freshly prepared mixture of potassium triiodide was then added dropwise to a solution of 5-iodoindole a (8.3 g, 24.0 mmol) and thiourea (2.2 g, 29.0 mmol) in MeOH (73 ml) at room temperature and stirred for 30 minutes. After filtration, the solution is concentrated under reduced pressure at 45° C., to half of its volume. To this mixture was added an aqueous solution of NaOH (9.6 ml, 10 N) which was heated to 95° C. for 30 minutes. Upon cooling to room temperature, a solution of ethyl bromobutyrate (4.2 ml, 29.0 mmol) in diethyl ether (50 ml) was added to the reaction mixture at room temperature and stirred vigorously for 45 minutes. After separation of the organic phase, the aqueous reaction mixture was additionally extracted with diethyl ether (100 ml) and then combined for additional washes with brine (50 ml) and dried over Na₂SO₄. The solvent was removed in vacuo to give a white solid, used directly in the next reaction. Lithium hydroxide (0.236 g, 5.64 mmol) was added to the crude ester (1.10 g, 2.82 mmol) dissolved in a 2:2:1 mixture of THF, EtOH and H₂O (14.2 ml) and heated to 50° C. for 2 hours. After cooling to room temperature, the reaction mixture was quenched with aqueous hydrochloric acid (12 ml, 1 M HCl) and extracted with diethyl ether (25 ml), washed with H₂O (20 ml), brine (20 ml) and dried over Na₂SO₄. The solvent was removed in vacuo, to afford b (0.968 g, 95%) as a clear oil.

c: To a solution of acid b (0.968 g, 2.68 mmol) in CHCl₃ (20 ml) was added polyphosphate ester (10 ml) and allowed to stir for 1.5 hours. The solution was then poured into ice water and extracted with EtOAc (3×20 ml). The organic layer was further washed with H₂O (20 ml), brine (20 ml) and dried over Na₂SO₄. The solvent was removed in vacuo, and the residue was subjected to flash chromatography (silica gel, 0→50% EtOAc in hexanes, gradient elution) to afford indole-ketone c (0.200 g, 21%) as a yellow solid.

d: To a cold (−78° C.) solution of indole-ketone c (0.200 g, 0.582 mmol) in THF (3 ml) was added LHMDS (1.8 ml, 1.0 M in THF) dropwise. After stirring for 1 hour at 0° C., pyruvonitrile (0.200 ml, 2.91 mmol) was added dropwise and the reaction mixture was allowed to stir for 30 minutes at −78° C. The reaction mixture was diluted with H₂O (5 ml) and extracted with EtOAc (3×5 ml). The organic layer was washed with brine (5 ml) and dried over Na₂SO₄. The solvent was removed in vacuo and the crude residue containing thiopyranoindole d was dissolved in EtOH (3 ml) and hydrazine (1 ml) and allowed to stir for 3 hours at room temperature. The reaction mixture was diluted with 0.1 M HCl (5 ml) and extracted with EtOAc (3×10 ml). The organic layer was washed with brine (5 ml) and dried over Na₂SO₄. The solvent was removed in vacuo and the residue subjected to flash chromatography (silica gel, 0→85% EtOAc in hexanes, gradient elution) to afford pyrazole e (66 mg, 30%) as a yellow solid.

9: A mixture of pyrazole e (44 mg, 0.12 mmol), CuI (1 mg, 0.006 mmol), Sodium methansulfinate (18 mg, 0.18 mmol), N,N-dimethylethlenediamine (1.27 μl, 0.012 mmol) was dissolved in DMSO (1 ml) and heated to 180° C. over 15 min by microwave. The reaction mixture was diluted with H₂O (1 ml) and extracted with EtOAc (3×2 ml). The organic layer was washed with brine (1 ml) and dried over Na₂SO₄. The solvent was removed in vacuo and the residue subjected to purification by HPLC, to afford 9 (6.1 mg, 16%).

### EXAMPLE 5

### synthesis of compound 3

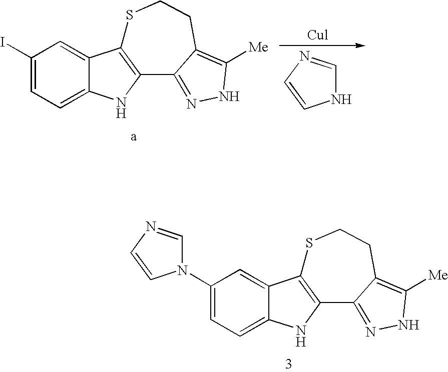

3: Compound a was combined with glycine (10 mg, 0.13 mmol), K₃PO₄ (140 mg, 0.66 mmol), CuI (10 mg, 0.026 mmol), imidazole (36 mg, 0.52 mmol) under N₂ and 1.3 ml of DMSO was added to this mixture. The reaction was subjected to microwave conditions at 170° C. for 30 minutes. The reaction mixture was filtered and directly subjected to purification by HPLC, to afford 45 mg of 3.

### EXAMPLE 6

### synthesis of compound 4

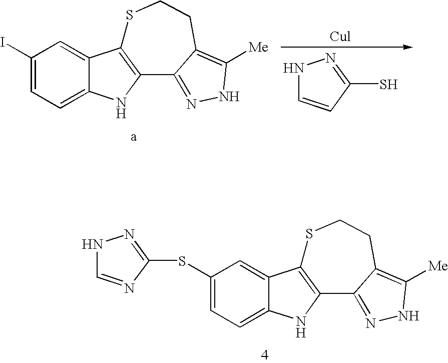

4: Using the procedures and conditions for preparing compound 3 compound a (50 mg) was reacted with 1H-1,2,4-triazole-3-thiol to give 10 mg of compound 4.

### EXAMPLE 7

### synthesis of compounds 5 and 6

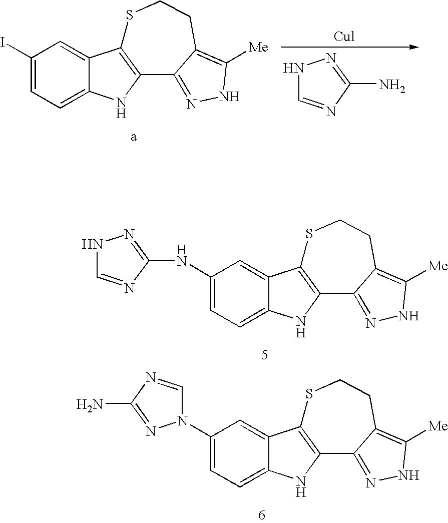

5 and 6: Using the procedures and conditions for preparing compound 3 compound a (50 mg) was reacted with 1H-1,2,4-triazol-3-amine to give 13 mg of compound 5 and 8 mg of compound 6.

### EXAMPLE 8

### Aurora A & Aurora B In Vitro Kinase Assays

Kinase activities were measured by Enzyme-Linked Immunosorbent Assay (ELISA): Maxisorp 384-well plates (Nunc) were coated with recombinant fusion protein comprising residues 1-15 of Histone H3 fused to the N-terminus of Glutathione-S-Transferase. Plates were then blocked with a solution of 1 mg/mL I-block (Tropix Inc) in phosphate-buffered saline. Kinase reactions were carried out in the wells of the ELISA plate by combining an appropriate amount of mutant Aur A and B kinases with test compound and 30 μM ATP. The reaction buffer was 1× Kinase Buffer (Cell Signaling Technologies) supplemented with 1 μg/mL I-block. Reactions were stopped after 45 minutes by addition of 25 mM EDTA. After washing, substrate phosphorylation was detected by addition of anti-phospho-Histone H3 (Ser 10) 6G3 mAb (Cell Signaling cat #9706) and sheep anti-mouse pAb-HRP (Amersham cat# NA93 IV), followed by calorimetric development with TMB.

### EXAMPLE 9

### Cellular Proliferation/Viability Assay

Potency of test compounds in inhibiting cellular proliferation and/or cellular viability was estimated using a cellular ATP assay (Cell-Titer-Glo, Promega). Cells (HCT116, HT29 colon cancer cell lines, MCF-7 breast cancer cell line) were seeded in 384-well plates (Greiner μClear) at an appropriate density in 50:50 DMEM/Hams F-12 medium supplemented with 10% fetal calf serum, and allowed to attach overnight. Test compounds were sequentially diluted in DMSO and then culture medium, and added to the cells at appropriate concentrations. Cells were incubated with compound for 5 days. Cell number/viability was estimated using Cell-Titer-Glo reagent (Promega) according to manufacturers instructions.

### EXAMPLE 10

### Cellular PhosphoHistone/Mitosis Assay

Efficacy of compounds in inhibiting progression through mitosis and Aurora B-dependent Histone H3 phosphorylation was estimated by automated microscopy and image analysis. HT29 colon cancer cells were seeded at an appropriate density in 384-well plates (Greiner μClear) in 50:50 DMEM/Hams F-12 medium supplemented with 10% fetal calf serum and allowed to attach overnight. Test compounds were sequentially diluted in DMSO and then culture medium, and added to the cells at appropriate concentrations. After 16 hours of incubation with compounds, cells were processed for immunofluorescent microscopy. Cells were fixed with 4% paraformaldehyde, then wells are blocked with 5% fish gelatin (Sigma), then incubated with anti-phospho-Histone H3 (Ser10) rabbit polyclonal antibody (Cell Signaling) and anti-MPM2 monoclonal antibody (Cell Signaling), followed by incubation with goat anti-rabbit-AlexaFluor 555 and sheep anti-mouse AlexaFluor 488 (Invitrogen) and nuclear counterstaining with Hoechst 33342. Images were acquired using a Discovery-1 automated microscopy system (Molecular Devices), and analyzed using MetaMorph software (Molecular Devices) to calculate the percentage of cells scoring positive for MPM2 and for Phospho-Histone H3.

We claim:

1. A compound of formula I: 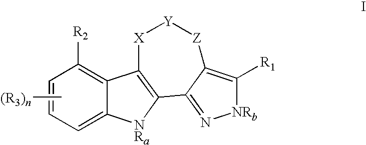
wherein
X, Y and Z are independently absent, CR₄R_{4′}, NR₅, S, SO, SO₂ or O; or X and Y together are CR₄═CR₄; or Y and Z together are CR₄═CR₄; wherein at least one of X, Y and Z is NR₅, S, SO, SO₂ or O;
Rₐ and R_{b} are independently H or a protecting group;
R₁ is H, hydroxyl, halogen, amino, or is alkyl, acyl, alkoxy or alkylthio optionally substituted with hydroxyl, halogen, oxo, thione, amino, carboxyl and alkoxy;
R₂ is H, halogen, hydroxyl, mercapto, amino, alkyl, a carbocycle or a heterocycle, wherein said alkyl, carbocycle and heterocycle are optionally substituted with halogen, hydroxyl, mercapto, amino, carboxyl, alkyl, a carbocycle or a heterocycle and wherein one or more CH₂ groups of an alkyl group is optionally replaced with —O—, —S—, —S(O)—, S(O)₂, —N(R₅)—, —C(O)—, —C(S)—, —C(O)—NR₅—, —NR₅—C(O)—, —SO₂—NR₅—, —NR₅—SO₂—, —NR₅—C(O)—NR₅—, —C(O)—O— or —O—C(O)—;
R₃ is halogen, hydroxyl, mercapto, amino, alkyl, a carbocycle or a heterocycle, wherein said alkyl, carbocycle and heterocycle are optionally substituted with halogen, hydroxyl, mercapto, amino, carboxyl, alkyl, a carbocycle or a heterocycle and wherein one or more CH₂ groups of an alkyl group is optionally replaced with —O—, —S—, —S(O)—, S(O)₂, —N(R₅)—, —C(O)—, —C(S)—, —C(O)—NR₅—, —NR₅—C(O)—, —SO₂—NR₅—, —NR₅—SO₂—, —NR₅—C(O)—NR₅—, —C(O)—O— or —O—C(O)—;
R4 and R₄′ are independently H, hydroxyl, halogen, amino, alkyl, a carbocycle or a heterocycle, or R₄ and R_{4′} together form oxo, thione, a carbocycle or heterocycle, wherein said alkyl, carbocycles and heterocycles are optionally substituted with halogen, hydroxyl, carboxyl, amino, alkyl, a carbocycle or a heterocycle and wherein one or more CH₂ groups of an alkyl group is optionally replaced with —O—, —S—, —S(O)—, S(O)₂, —N(R₅)—, —C(O)—, —C(O)—NR₅—, —NR₅—C(O)—, —SO₂—NR₅—, —NR₅—SO₂—, —NR₅—C(O)—NR₅—, —C(O)—O— or —O—C(O)—;
R₅ is H, alkyl, a carbocycle or a heterocycle wherein one or more CH₂ or CH groups of said alkyl is optionally replaced with —O—, —S—, —S(O)—, S(O)₂, —NH—, or —C(O)—; and said alkyl, carbocycle and heterocycle is optionally substituted with hydroxyl, alkoxy, acyl, halogen, mercapto, oxo, carboxyl, acyl, halo-substituted alkyl, amino, cyano nitro, amidino, guanidino an optionally substituted carbocycle or an optionally substituted heterocycle;
n is 0 to 3;
and salts and solvates thereof.

2. The compound of claim 1, wherein X is CR₄R_{4′}, Y is S and Z is CR₄R_{4′}.

3. The compound of claim 2, wherein R₄ and R_{4′} are each H.

4. The compound of claim 1, wherein X is CR₄R_{4′}, Y is NR₅, and Z is CR₄R_{4′}.

5. The compound of claim 4, wherein R₄ and R₄ are each H and R₅ is alkyloxycarbonyl.

6. The compound of claim 1, wherein X is S, Y is CR₄R_{4′}, and Z is CR₄R_{4′}.

7. The compound of claim 6, wherein R₄ and R_{4′} are each H.

8. The compound of claim 1, wherein Rₐ and R_{b} are both H.

9. The compound of claim 1, wherein R₁ is alkyl.

10. The compound of claim 1, wherein R₁ is methyl.

11. The compound of claim 1, wherein R₂ is H.

12. The compound of claim 1, wherein R₃ is methoxy, methylsulfonyl, 1H-imidazol-1-yl, 1H-1,2,4-triazol-3-yl-thio, 1H-1,2,4-triazol-3-yl-amino, 3-amino-1H-1,2,4-triazol-1-yl or 1-hydroxy-1-(5-methylfuran-2-yl)methyl.

13. The compound of claim 1, wherein n is 1.

14. The compound of claim 1, wherein n is 1 and R₃ is methoxy, methylsulfonyl, 1H-imidazol-1-yl, 1H-1,2,4-triazol-3-yl-thio, 1H-1,2,4-triazol-3-yl-amino, 3-amino-1H-1,2,4-triazol-1-yl or 1-hydroxy-1-(5-methylfuran-2-yl)methyl.

15. The compound of claim 1, wherein n is 1; Rₐ and R_{b} are both H; R₁ is alkyl; R₂ is H; is methoxy, methylsulfonyl, 1H-imidazol-1-yl, 1H-1,2,4-triazol-3-yl-thio, 1H-1,2,4-triazol-3-yl-amino, 3-amino-1H-1,2,4-triazol-1-yl or 1-hydroxy-1-(5-methylfuran-2-yl)methyl.

16. The compound of claim 15, wherein X is CR₄R_{4′}, Y is NR₅, and Z is CR₄R_{4′}, and R₄ and R₄ are each H and R₅ is alkyloxycarbonyl.

17. The compound of claim 1 selected from the group consisting of:

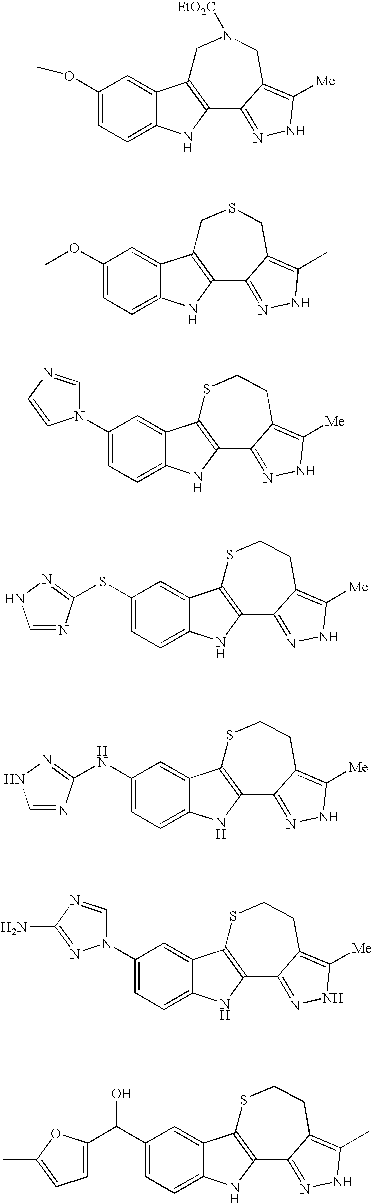

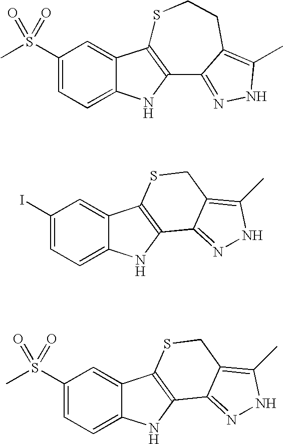

18. A method for inhibiting the signaling of an Aurora kinase in a cell comprising contacting said Aurora kinase with a compound of claim 1.

19. A method for treating a disease or condition in a mammal associated with the signaling of an Aurora kinase, comprising administering to said mammal an effective amount of a compound of claim 1.

20. A method for treating cancer, comprising administering to said mammal an effective amount of a compound of claim 1.
